(19) United States
(12) Plant Patent Application Publication
(10) Pub. No.: US 2015/0327421 P1
(43) Pub. Date: Nov. 12, 2015

(54) VERBENA PLANT NAMED 'KLEVP12427'

(50) ;

(71) Applicant: Klemm+Sohn GmbH & Co. KG, Stuttgart (DE)

(72) Inventor: Ruijun Li, North Parramata (AU)

(73) Assignee: Klemm+Sohn GmbH & Co. KG, Stuttgart (DE)

(21) Appl. No.: 14/120,222

(22) Filed: May 7, 2014

(51) Int. Cl. A01H 5/00

(52) U.S. Cl. PLT/308

(57) ABSTRACT

A new Verbena plant particularly distinguished by large, red-purple flowers with white centers, a semi-upright plant habit, good tolerance to powdery mildew, and early flowering, is disclosed.

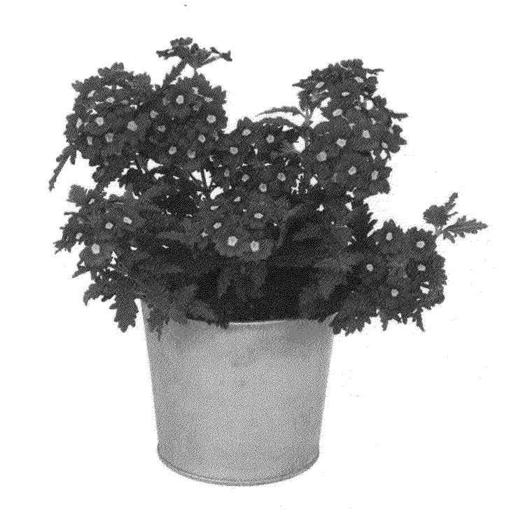

### CROSS-REFERENCE TO RELATED APPLICATIONS

A Canadian Plant Breeders Rights application was filed on Feb. 25, 2014, application number 14-8204. ‘KLEVP12427’ has not been sold or made publicly available more than one year prior to the filing date of the present application.

### GENUS AND SPECIES

Verbena sp.

### VARIETY DENOMINATION

‘KLEVP12427’

### BACKGROUND OF THE NEW PLANT

The present invention comprises a new and distinct variety of Verbena, botanically known as Verbena sp., and hereinafter referred to by the variety name ‘KLEVP12427’. ‘KLEVP12427’ originated from a cross-pollination conducted in September 2008 in Sydney, Australia between the proprietary female Verbena variety ‘13 112’ (unpatented), and the proprietary male Verbena variety ‘V 842’ (unpatented).

The seeds from the cross were sown and plants were grown for evaluation, where an individual plant designated ‘KLEVP12427’ was selected from the group of plants in Cobitty, NSW, Australia in April 2009.

In April 2009 ‘KLEVP12427’ was first vegetatively propagated by vegetative cuttings in Cobitty, NSW, Australia. ‘KLEVP12427’ was found to reproduce true to type in successive generations of asexual propagation via vegetative cuttings.

### SUMMARY

The following are the most outstanding and distinguishing characteristics of this new variety when grown under normal horticultural practices in Stuttgart, Germany.

1. Large, red-purple flowers with white centers;

2. A semi-upright plant habit;

3. Good tolerance to powdery mildew; and

4. Early flowering.

### DESCRIPTION OF THE PHOTOGRAPH

This new Verbena plant is illustrated by the accompanying photograph which shows the plant's overall plant habit including form, foliage, and flowers. The photograph is of a two to three-month old plant grown in a greenhouse in Stuttgart, Germany in May 2012. The colors shown are as true as can be reasonably obtained by conventional photographic procedures.

### DESCRIPTION OF THE NEW VARIETY

The following detailed descriptions set forth the distinctive characteristics of ‘KLEVP12427’. The data which define these characteristics were collected from asexual reproductions carried out in Stuttgart, Germany. Data was collected on three-month-old plants grown in a greenhouse in Stuttgart, Germany in February 2014. Color references are to The R.H.S. Colour Chart of The Royal Horticultural Society of London (R.H.S.) 2005.
Classification:
   Family.—Verbenaceae.
   Botanical.—Verbena sp.
   Common.—Verbena.
   Designation.—‘KLEVP12427’.
   Parentage:
   Female parent.—The proprietary female verbena variety ‘13 112’ (unpatented).
   Male parent.—The proprietary male verbena variety ‘V 842’ (unpatented).
Plant:
   Growth and branching habit.—Semi-upright.
   Height (from top of soil).—30.0 cm.
   Width (horizontal plant diameter).—30.0 cm.
   Time to produce a finished flowering plant.—Approximately 8 weeks.
   Time to initiate and develop roots.—3 weeks.
   Root description.—White.
Stems:
   Number of branches per plant.—More than 40.
   Growth habit.—Semi-upright.
   Length.—20.0 cm.
   Diameter.—1.0 cm.
   Internode length.—Approximately 2.0 cm.
   Color.—RHS 130D.
   Anthocyanin.—Absent.
   Texture and appearance.—Pubescence, round.
Leaves:
   Arrangement.—Opposite.
   Immature and mature leaf color.—Upper surface: RHS 141A Lower surface: RHS 141C.
   Length.—2.5 cm.
   Width.—1.5 cm.
   Shape.—Lanceolate.
   Apex.—Acute.
   Base.—Obtuse.
   Margin.—Crenate.
   Texture.—Upper surface: Leathery Lower surface: Rugose.
   Venation type.—Pinnate.
   Venation color (both upper and lower surfaces).—RHS 141D.
   Petioles.—Length: 1.0 cm Diameter: 0.1 cm Color: RHS 141D Texture: Leathery.
Flower buds:
   Shape.—Tall, cylindrical.
   Length.—0.5 cm.
   Diameter.—0.1 cm.
   Color (at tight bud).—RHS 74C.
Inflorescence:
   Blooming habit (flowering season).—Continuously.
   Lastingness of individual blooms on the plant.—2 weeks.
   Fragrance.—Absent.
   Inflorescence type.—Umbel.
   Length of inflorescence.—6.0 cm.
   Diameter of inflorescence.—6.0 cm.
   Number of florets per inflorescence.—More than 15.
   Peduncle.—Color: RHS 141A Length: 1.0 cm Diameter: 0.1 cm.
Florets:
   Length.—2.0 cm.
   Diameter.—4.0 cm.
   Texture.—Glossy.
   Form.—Umbel.
   Length.—0.1 cm.
   Diameter.—4.0 cm.
   Color.—Immature: Upper surface: RHS 74A Lower surface: RHS 74B Mature: Upper surface: RHS 74B Lower surface: RHS 74C.
   Corolla tube length.—0.2 cm.
   Corolla tube color.—RHS 74C.
   Petals.—Length of lobe: 2.0 cm Diameter of lobe: 2.0 cm Lobe shape: Round Apex: Acute Base: Obtuse Margin: Entire Texture (both upper and lower surfaces): Glossy, satiny.
   Pedicels.—Length: 2.5 cm Diameter: About 0.1 cm Color: RHS 74C Texture and appearance: Hairy, dull Arrangement: Round.
Sepals:
   Shape.—Lanceolate.
   Apex.—Acute.
   Base.—Obtuse.
   Margin.—Entire.
   Length.—2.0 cm.
   Diameter.—0.5 cm.
   Color.—RHS 141C.
   Texture (both upper and lower surfaces).—Hairy, dull.
Reproductive organs:
   Stamens.—Present.
   Quantity.—Numerous.
   Filament length.—0.1 cm.
   Filament diameter.—Less than 0.1 cm.
   Anther.—Shape: Elliptic Color: RHS 141D Length: Less than 0.1 cm Diameter: Less than 0.1 cm.
   Pollen amount.—Absent.
   Pistils.—Number: 1 Length: 1.0 cm Diameter: Less than 0.1 cm Stigma: Color: RHS 141D Appearance: Ovate Length: Less than 0.1 cm Diameter: Less than 0.1 cm Style: Color: RHS 141D Length: 0.2 cm Diameter: Less than 0.1 cm Appearance: Long.
Fruit and seed set: None observed.
Disease and pest/insect resistance: Good resistance to powdery mildew.

### COMPARISON WITH PARENTAL LINES AND KNOWN VARIETY

‘KLEVP12427’ is distinguished from its parents as described in Table 1.

TABLE 1
Comparison with Parental Lines

| | Variety | Female parent | Male Parent |
|---|---|---|---|
| Characteristic | ‘KLEVP12427’ | ‘13 112’ | ‘V 842’ |
| White eye | Big | Very small | Big |
| (flower center) | | | |
| Earliness to flower | Early | Medium | Very early |

‘KLEVP12427’ is most similar to the commercial verbena ‘Empress Purple’ (patent status unknown). Differences between the varieties are described in Table 2.

TABLE 2
Comparison with Similar Variety

| | Variety | Commercial line |
|---|---|---|
| Characteristic | ‘KLEVP12427’ | ‘Empress Purple’ |
| Flower size | Bigger than | Smaller than |
| | ‘Empress Purple’ | ‘KLEVP12427’ |
| Earliness to flower | Earlier than | Later than |
| | ‘Empress Purple’ | ‘KLEVP12427’ |
| Resistance to | Good resistance | Low resistance |
| Powdery Mildew | | |

I claim:

1. A new and distinct variety of Verbena plant designated ‘KLEVP12427’ as shown and described herein.
